(19) United States
(12) Patent Application Publication
(10) Pub. No.: US 2025/0130706 A1
(43) Pub. Date: Apr. 24, 2025

(54) SYSTEMS AND METHODS FOR DIRECT INTEGRATION OF A PREVIEW FUNCTION

(71) Applicant: ICON CLINICAL RESEARCH LIMITED, Dublin (IE)

(72) Inventors: Jeff BEELER, Cary, NC (US); Matt SABOL, Springfield, VA (US); John BIGLEY, Lincoln, CA (US)

(21) Appl. No.: 18/680,113

(22) Filed: May 31, 2024

Related U.S. Application Data
(60) Provisional application No. 63/594,356, filed on Oct. 30, 2023.
(60) Provisional application No. 63/591,391, filed on Oct. 18, 2023.

(51) Int. Cl. G06F 3/04847; G06F 3/0481

(52) U.S. Cl. CPC G06F 3/04847; G06F 3/0481

(57) ABSTRACT

A computer-implemented method for providing a device-agnostic environment with real-time rendering during instrument construction may include causing, via a processor of a user device, a graphical user interface of the user device to output a construction interface operable to edit contents of an instrument; causing, via the processor, the graphical user interface to output a rendered view of the instrument, wherein: the rendered view is generated via a simulated rendering environment of a base device operating on the user device, such that the simulated rendering environment interprets data of the instrument and generates the rendered view as if the instrument were natively displayed on the base device and while being agnostic to display characteristics of the user device; and the simulated rendering environment is configured to update the rendered view in real-time in response to adjustment to the construction interface.

### CROSS-REFERENCE TO RELATED APPLICATION

The present application claims benefit from U.S. Provisional Patent Application 63/594,356 filed on Oct. 30, 2023, which claims benefit from U.S. Provisional Patent Application US 63/591,391 filed on Oct. 18, 2023, the entirety of which are incorporated herein by reference.

### FIELD

Various embodiments of the present disclosure relate generally to providing a device-agnostic environment for interacting with interactive documents with real-time rendering, and more specifically to systems and methods for simulated rendering of interactive documents as if natively displayed by a base device.

### BACKGROUND

Generally, clinical research involves standardization of clinical endpoints. Clinical endpoints may refer to the outcome of a clinical trial that may be statistically analyzed to help determine the efficacy of the study performed within the clinical research. Standardization of the clinical endpoint may assist in quantifying specific measures to support changes in reported data. An exemplary clinical endpoint may be questionnaires that have been validated clinically to support a regulatory submission. Building a validated questionnaire or similar product may be complex and time consuming, as can implementing and managing the administration of such clinical tools.

Conventional techniques generally require validating a particular questionnaire for each study the questionnaire is utilized in, and the development of a clinical data collection tool is required to meet certain predefined standards, including standards based on how the questions or related materials are presented to a participant user. Accurately previewing how an instrument will appear and function on different devices both during the development process and when providing an instrument to a participant or other user can be challenging,

Conventional Systems and approaches may require separate instances for each device, leading to increased computational overhead and complexity. Such techniques often lack the flexibility to adapt to different display characteristics, necessitating device-specific coding and libraries. Other approaches, on the other hand, may be bandwidth-intensive and may introduce performance and latency issues, especially for complex applications.

Moreover, many conventional solutions may be time-consuming to set up, hindering the iterative development process. In the context of clinical trial applications, such as electronic clinical outcome assessment (eCOA) instruments, the need for accurate and efficient previewing becomes even more critical due to the stringent regulatory requirements and the potential impact on patient safety.

The present disclosure is directed to addressing challenges such as those referenced above. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

In one aspect, a computer-implemented method for providing a device-agnostic environment with real-time rendering during instrument construction may include: via a processor of a user device, causing a graphical user interface of the user device to output a construction interface operable to edit contents of an instrument; and via the processor, causing the graphical user interface to output a rendered view of the instrument, wherein: the rendered view is generated via a simulated rendering environment, operating on the user device, of a base device such that the simulated rendering environment interprets data of the instrument and generates the rendered view as if the instrument were natively displayed on the base device and while being agnostic to display characteristics of the user device; and the simulated rendering environment is configured to update the rendered view in real-time in response to adjustment to the construction interface.

In another aspect, a computer-implemented method for providing a device-agnostic environment for displaying validated instruments may include: via a processor of a user device, receiving an identification of a trial associated with a user of the user device; via the processor, identifying a validated instrument corresponding to the trial and to the user by accessing a remote system; via the processor, obtaining data of the validated instrument from the remote system; via the processor, causing a graphical user interface of the user device to output a rendered view of the validated instrument based on the obtained data, wherein the rendered view is generated via a simulated rendering environment, operating on the user device, of a base device such that the simulated rendering environment interprets data of the validated instrument and generates the rendered view as if the validated instrument were natively displayed on the base device and while being agnostic to display characteristics of the user device; receiving, via the processor and by the graphical user interface, user input into the rendered view of the validated instrument; and via the processor, transmitting the user input to the remote system.

In a further aspect, a computer-implemented method for providing a device-agnostic environment for displaying instruments may include: via a processor of a user device, obtaining data of an instrument; via the processor, executing a simulated rendering environment that simulates a native display of a base device while being agnostic to display characteristics of the user device; via the processor, causing a graphical user interface of the user device to output, using the simulated rendering environment, a rendered view of the instrument based on the obtained data, such that the instrument is displayed as if the instrument were natively displayed on the base device.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for creating, validating, and administering an instrument, according to one or more embodiments.

FIG. 2 depicts an exemplary flowchart of a patient accessing a study, according to one or more embodiments.

FIG. 3 depicts a dependency stack of the previewer, according to one or more embodiments.

FIG. 4 depicts a dependency diagram of the previewer, according to one or more embodiments.

FIG. 5A depicts an exemplary method for providing a device-agnostic environment with real-time rendering during instrument construction, according to one or more embodiments.

FIG. 5B depicts an exemplary method for providing a device-agnostic environment for displaying validated instruments, according to one or more embodiments.

FIG. 6 depicts an exemplary flow diagram illustrating a workflow for obtaining an instances of a validated instrument, according to one or more embodiments.

FIG. 7 depicts an exemplary flow diagram illustrating a workflow for obtaining access to a validated instrument, according to one or more embodiments.

FIG. 8 depicts a simplified functional block diagram of a computer, according to one or more embodiments

### DETAILED DESCRIPTION OF EMBODIMENTS

Various embodiments of the present disclosure relate generally to a previewer tool, and more specifically to systems and methods for direct integration of a preview function.

According to embodiments disclosed herein, systems and methods are described for providing a device-agnostic environment for displaying instruments. A clinical trial or similar operation may have corresponding instruments such as an electronic clinical outcome assessment (“eCOA”).

As discussed above, conventional techniques of streaming devices to integrate their display or spinning up virtual machines may be computationally complex and difficult to scale. Thus,, in one aspect, there is a need for a more efficient and device-agnostic solution that would enable developers to preview applications in real-time during construction, accurately representing how they would appear on various target devices, and also enable a consistent user experience during a clinical trial, regardless of the particulars of a user's device. Such a solution would streamline the development process, improve the quality of the final product, and reduce the risk of errors or inconsistencies across different platforms.

The systems and methods disclosed in the present invention offer several advantages over conventional systems for reviewing and accessing applications. The direct integration of the preview function eliminates the need for separate devices or emulation instances, reducing computing costs and complexity. Additionally, such a previewer tool may be built on or utilize the same production environment as the development application, streamlining development and ensuring consistency between the preview and actual user experience.

In some embodiments, a simulated rendering environment enables achieving device agnosticism, and the device-agnostic nature of the rendering environment ensures accurate representation of instruments across various devices, without requiring device-specific coding or libraries. Such an approach may include a rendering engine or the like that interprets the instrument's data and generates the rendered view as if the instrument were natively displayed on the base device, regardless of the display characteristics of the user device, ensuring a consistent user experience across different devices. Moreover, real-time (e.g., near real-time) rendering capabilities may provide intuitive feedback during instrument construction, and enable seamless interaction with validated instruments.

Reference to any particular activity is provided in this disclosure only for convenience and not intended to limit the disclosure. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if”' is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a patient, e.g., to provide medical care, medical intervention or advice, or the like. Terms like “patient,” “client,” “participant,” “study participant” or the like generally encompass any person or entity who is obtaining information, seeking resolution of an issue, or engaging in any other type of interaction with a medical provider, e.g., to receive such care, information, or the like. Terms like “user” generally encompass any person or entity that may obtain information, resolution of an issue, purchase of a product, or engage in any other type of interaction with a provider or patient, whereby the user may be the medical provider or the patient as the case may be. For example, an individual that collects data on themselves may be both a patient and a user. A user may also refer to a patient that is being assisted by a provider. Further, a user may refer to a provider entering data on behalf of a patient.

In some instances, implementing a clinical trial to conduct clinical research may include having standardization of clinical endpoints. The clinical endpoints may include outcome measures of clinical research trials. Clinical endpoints may include instruments or artifacts. An instrument or artifact may refer to an assessment that has been developed that has been shown to provide clinical meaningful outcomes based upon a certain change from the baseline of a participant. An instrument may contain specific structure and may be configured to be displayed in a manner specific to an author's intent and/or clinical trial guidelines or regulations. An artifact may include any other material or item with textual content that may be provided as part of a clinical trial, e.g., a schedule, disclosure, etc. While examples and embodiments below may reference an instrument or an artifact, it should be understood that the techniques disclosed herein may be adapted to either, as well as to any other suitable textual content.

An exemplary instrument may be a questionnaire such as an electronic clinical outcome assessment (“eCOA”). An eCOA may need to be validated clinically to support regulatory submissions. Clinical validation may be a process of verifying an instrument's procedures (e.g., the set of questions) to verify that the measurements provide a clinically meaningful outcome. A clinical validation may, for example, be performed internally by a particular organization. A clinical validation may be constructed to be standardized for a validated instrument (e.g., such as a validated eCOA). A clinical validation may promote the use of a standard set of data from time point to time point and provide reliable results with a known outcome that is based upon a change over time as opposed to a random error.

The process of creating, validating, and using an instrument (e.g., an eCOA) may be time consuming. For example, the process of performing a clinical validation may take multiple months for a single eCOA to be created and validated.

Conventional systems for developing an instrument such as an eCOA may be very time consuming and complex. In an example, validation may require generating screenshots or views of the instrument in the environment in which it will be utilized, e.g., on a user device. Conventionally, this would entail assembling the data that drives the rendering of an eCOA (e.g., data objects defining questions and responses), and spinning up a native environment for the desired output (e.g., a virtual machine simulation of a device, an actual device used directly and/or streamed to a developer machine), and observing and recording how the eCOA is displayed. The rendering operations above may result in time consuming and complex steps between making a change to the eCOA and being able to view the rendered result. As such, iterations and small changes, which are common during the development process, may be very difficult and time consuming to make.

One or more embodiments of the system described herein may provide a digital platform that has the means to configure a clinically validated instrument, create screenshots for verification from the author, and store a digital version for use without requiring spinning up a virtual machine or separate instance of an output device. The digital platform may support various features such as, for example, digital storage of an instrument, certification of validations of an instrument, translations of a particular instrument, screenshots of a particular instrument, and means to utilize a digital store for an identical representation of a validated instruments for one or more studies.

One or more embodiments of the system may offer the advantage of only requiring a single configuration of an instrument. For example, the system may offer tools to create and store a particular eCOA within the platform. The system may offer the advantage of requiring only a single validation of a created instrument. This instrument may be accessed and utilized in multiple studies while staying validated, as will be described in greater detail below. The system may offer a single certification of an instrument. Certification as used herein generally encompasses a process by which an instrument undergoes a technical validation that it conforms to an author's original electronic or paper version according to the author's wishes. This technical validation process, in embodiments, may be separate from a clinical validation of the instrument (i.e., confirming that endpoints are statistically significant), and instead refers to the verification that the look and interpretation is as the author intended with the original validated instrument.

One or more embodiments of the system may offer tools for a user and/or an organization to prepare an eCOA. The system may further offer tools to create a study, as well as a schedule that utilizes a validated eCOA. A study may refer to a research study involving human subjects that aims to evaluate the effects of a biomedical intervention (e.g., drugs, treatment, devices, etc.) on health-related outcomes. A schedule may refer to a schedule of activities that provides the tasks and milestones, along with the corresponding timeline that are critical to the execution of a study.

One or more embodiments of the system may offer a digital platform that utilizes a method for using data reference links to maintain data validation during use. As will be discussed in greater detail below, a validated instrument may be accessed for a trial session during a study. The system may allow for the transmission of a request for a particular instance of a validated instrument during the trial session. The system may be configured to receive a data reference object that is operable as a data reference link to the validated instrument that is stored in an instrument library, such that the data reference object is operable to execute the validated instrument without generating a copy of the validated instrument. The system may further allow for a Graphical User Interface (“GUI”) of the trial session to operate the data reference object, such that the user device executes the validated instrument by reference.

Conventional systems may utilize an emulator or stream to review and access aspects of an application used to host data and/or server user interactions, such as an application to receive user responses to validated instruments for clinical studies. One or more emulators may be hosted, e.g., via a virtual machine, with each emulator operating as a virtual environment for a user device. In another example, a screen output from a device may be streamed from the device to a system used for review and/or access, and interactions from the system may be streamed back as input. Utilizing a virtual machine or stream may be extremely time and data consuming for a system. For example, each instance of access and/or review requires either a separate device to stream from or a separate emulation instance. In other words, the need for a one-to-one relationship between devices/emulations and system instances means that these conventional solutions have a computing cost and complexity that scales with concurrent use. Further, the unique circumstances of some applications, such as handling eCOA interactions for a clinical trial, may add to these costs of scale, which may require extensive processing power and be slow and costly. Further, emulators emulate a particular device, and thus are not a one-size-fits all solution. In order to accurately portray how an application may appear on different devices, many emulators may be required, which may require device-specific coding, libraries, or other computing complexities in order to account for variations in emulation. Moreover, initiating an emulation or stream may be a time-intensive process. Creating new instances or setting up a stream on a device may take, for example, five to ten minutes of time per instance. In an iterative development environment in which such creation may occur frequently, such time delays may interruption and negatively impact the development process. Thus, an emulator and/or stream may not be scalable or optimal for one or more of the operations described herein.

One or more embodiments may include the direct integration of a preview function built into the systems described herein. The preview function may, for example, include or simulate the same engine that is utilized by the system to run a validated instrument (e.g., to access and fill out an eCOA library). In other words, rather than emulating a device or streaming the output of a device, direct integration effectively natively generates output of an application on a system as it would be displayed on a desired device. Generating a rendered view via a simulated rendering environment of a base device maintains device agnosticism, improving accurate representation of the instrument. In some embodiments, the device is, as example, a mobile device, and the rendered view provides the instrument as it would appear on an interface for a mobile device. For instance, a base device may be selected as the basis for consistent display and interaction. The interface may be a GUI, and in certain embodiments, the interface is a web interface for a user device. In additional embodiments, user input for interactions with a rendered view are received via a GUI, and a processor transmits this input to a remote system, facilitating interaction with the validated instrument.

The previewer may, for example, be configured to enable a user to view the content of the application through a computer interface of a computer system, in which the computer interface includes a preview as would be displayed by a mobile phone interface. In one aspect, the displayed preview is interactive (e.g., via a frontend with users and/or via a backend with other systems, devices, the internet, etc.) In an embodiment, the previewer may function as a “player” for the application. This not only enables iterative and concurrent use without initiating an emulation or stream, but also the same “player” used for development may also be used to render actual use of the application by users on mobile devices. In other words, in some cases, the previewer tool may be built on or utilize the same production environment as the application, reducing a need for separate development of developer tools or the like.

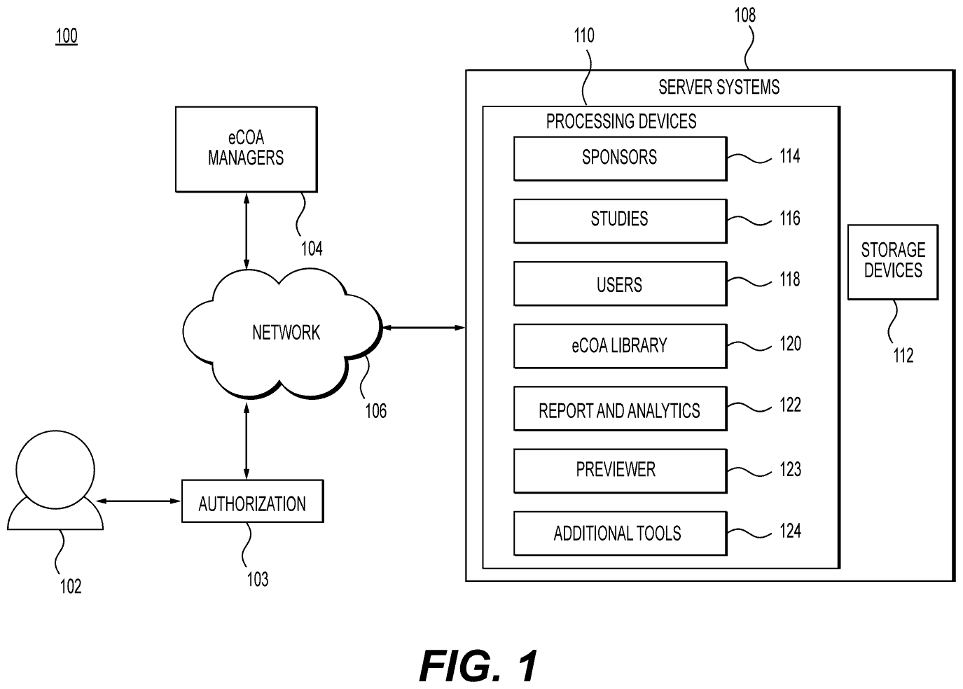

FIG. 1 depicts an exemplary environment for creating, validating, and administering an instrument, according to one or more embodiments. Environment 100 depicts an electronic network 106 that may be connected to systems associated with or operating as an eCOA managers 104. An eCOA manager 104 (as used herein generally encompassing any system personnel associated with developing and/or administering a trial), may be associated with a server configured to interact with the server systems 108 described below. The eCOA manager 104 may be operable for creating, certifying, and/or validating instruments for one or more studies. An eCOA manager 104 may further be utilized to create a study as well as a schedule for a study. The eCOA manager 104 may further include a trial server to initiate a trial session of a clinical study for one or more users 102.

Further, a user 102 may access one or more instruments through the network 106. A user 102, may for example be a patient participating within one or more studies. The user 102 may speak a language associated with the clinical study (e.g., a primary language), or a different language that the clinical study is conducted in (e.g., a secondary language). The user 102 may, for example, access an eCOA for a particular study through a GUI of a user device. The user device may be a computing device such as computer 700 described below. This may allow a user 102 to respond to and answer questions for a particular eCOA. The eCOA forms may be located in an eCOA library 120 of a server systems 108 as will be discussed in more detail below. When a user 102 accesses the system described herein, a unique token may be created that allows for the system to uniquely identify the user 102. The token may include metadata (e.g., in the standard of ISO 639-1 standard language code) of the respective user's 102 preferred language.

A user 102 may require an authorization from an authorization system 103 prior to accessing an eCOA form. For example, a user 102 may first require permission to access eCOA forms. As will be discussed in further detail below, eCOA forms may be stored in a fast healthcare interoperability resource (“FHIR”) server. FHIR may refer to the set of rules and specifications required for exchanging electronic healthcare data. To access the FHIR server, the user 102 may utilize an authorization system 103, as the FHIR server may not be accessible directly from the internet. The authorization system 103 may include an identity provider (“IDP”) web server that allows access to the FHIR server in which the eCOA forms are stored. The authorization system 103 may further include a firewall and a particular application programming interface (“API”) (e.g., an Azure API for FHIR) for accessing the eCOA forms stored in the eCOA library 120.

A user 102, e.g., a participant, administrator, or the like, either at a clinical site or remotely may utilize a computing device (e.g., computer 700) to obtain access to a particular study. The computing device may be represented as a “client-side caller” device. This device may identify whether the participant has access to a particular study service, e.g., by performing a permission call. Permission may refer to an identifier for resources that client wants to access. The permission call may have access to a local device conducting a study. The local device may for example have access to server systems 108.

According to an exemplary embodiment of the present disclosure, the electronic network 106 may also be connected to server systems 108, which may include processing devices 110 that are configured to implement one or more modules or servers (e.g., sponsors module 114, studies module 116, users module 118, eCOA library 120, report and analytics module 122, previewer 123, and additional tools 124 configured to create, certify, validate, administer, and or store one or more instruments for use within a study, according to an exemplary embodiment of the present disclosure.

The sponsors module 114 may be configured to store information related to a sponsor utilizing the server systems 108 to conduct a study. For example, the sponsors module 114 may keep track of organizations that are utilizing the server systems 108 to conduct a study. The sponsors module 114 may track each sponsor's studies, plans, and any created instruments by the particular sponsor.

The studies module 116 may include a database tracking all active and past studies performed by the server systems 108. The database within the studies module 116 may include, for example, a protocol number, a study name, a corresponding sponsor, a configuration status, and any pending actions for any studies conducted by the server systems 108. A moderator for a study may access the current results of a study through the studies module 116.

A users module 118 may be configured to store information related to patients of a study, study personnel, and system administrator. The users module 118 may further include any studies the user is participating in. The users module 118 may further include a user's full name, language(s) spoken, email address, role within the system, account status, and last login into the system described herein. Further, the users module 118 may be configured to add additional users to one or more studies. For example, an eCOA manager 104 may enter the email address, name, language, time zone, role, assigned study, and assigned site of a new user to the users module 118.

The eCOA library 120 may be configured to store one or more instruments including an eCOA. The eCOA library 120 may store the title, type of instrument, status of the instrument, amount of external version, amount of internal versions, and allow for actions to be conducted regarding an instrument. The eCOA library 120 and/or documents within the eCOA library 120 may be stored in a FHIR server. The eCOA library 120 may further include a “new form” function. The new form function may include a graphical user interface configured to enable an individual (e.g., an eCOA manager 104) to create a new eCOA form. For example, the interface may allow a user to create a display type, questions types, and sliders for a particular study. Question types may include the number, quantity, date, time, text, choice, and EuroQol (EQ)-visual analogue scale (VAS). The eCOA library 120 may further be configured to store a translation key, the translation key defining an arrangement of source text within a validated instrument.

The reports and analytics module 122 may be configured to store one or more reports and or analytics corresponding to one or more studies from the studies module 116. The reports and analytics library may include one or more data analysis tools to further analyze, graph, and organize responses to the instruments from the eCOA library 120. Further, reports may be generated that include the analysis and graphs performed. The analytics and reports may for example be accessed by the eCOA managers 104.

The previewer 123 may further include the ability to provide a preview element for an eCOA manager 104 preparing an instrument. This may enable a configurator to render an emulated version of a clinical eCOA without deploying it to a separate environment and loading it into a mobile device. This may expedite the process of developing an instrument. A problem with conventional clinical questionnaires is that the process for preparing questionnaires generally includes a step to render and review screenshots of the instrument. This may be cumbersome as the instrument may need to be deployed to an environment and rendered on the mobile device multiple times before getting spacing, text, and font completed to the author's specification. The system described herein may offer advantages as compared to conventional systems, such as: (1) a configured screen can be reviewed for accuracy, completeness, and overall design without leaving the configuration tool during construction of an instrument; (2) screenshots can be taken via the tool to reduce the need for one-by-one screenshots rendered on a mobile device; and (3) configuration may become quicker without deployments to a separate environment for verification. Advantage (2) may take effect during the certification process of an instrument, and subsequent submission to an internal review board. In some instances, one or more screenshots of each instrument may be required as evidence of the representation of the instrument on a mobile device. Each screenshot may then be used to confirm the instrument conforms to the standards set by the author and does not coerce or lead the participant in any way based upon the layout, button placement, etc. Overall, this feature may assist with expediting the process of creating an instrument.

The additional tools 124 may include one or more processors or servers configured to perform function such as: developing a study, a planner for determining a schedule for a particular study, a study configuration, translations, sites, tokenization, and resources. Study configuration may refer to the phases, visits, recurring questionnaires, and their association with each. Configuration may refer to “how” instruments are assigned in a specific schedule for a participant or clinical site to complete the questionnaire. The execution of the study schedule may refer to “when” each of the events (e.g., questionnaires) will occur. The study configuration may include a calendar of the particular events. Sites may refer to the clinical sites that are defined for each study. Each site may have one or multiple languages assigned to the configuration. The developing a study function may allow for a user to upload planned medical tests and measurements, as well as a particular timelines for all parts of the clinical study. Once a study is created, a user (e.g., an individual administering or creating the study) may assign a timeline for each phase of the study.

The server systems 108 may further include one or more storage devices 112. The storage devices 112 may be responsible for storing uploaded instruments, studies, clinical trial information, user information, reports and analytics, as well as sponsor information. Each of the modules within processing devices 110 may have access to the one or more storage devices 112.

Any of the above devices, tools and modules may be located on a device that may be connected to an electronic network 106, such as the Internet or a cloud service provider, through one or more computers, servers, and/or handheld mobile devices.

The additional tools 124 may include one or more processors or servers configured to perform function such as: developing a study, a planner for determining a schedule for a particular study, a study configuration, translations, sites, tokenization, and resources. Study configuration may refer to the phases, visits, recurring questionnaires, and their association with each. The study configuration may include a calendar of the particular events. Sites may refer to the clinical sites that are defined for each study. Each site may have one or multiple languages assigned to the configuration. The developing a study function may allow for a user to upload planned medical tests and measurements, as well as a particular timelines for all parts of the clinical study. Once a study is created, a user (e.g., an individual administering or creating the study) may assign a timeline for each phase of the study.

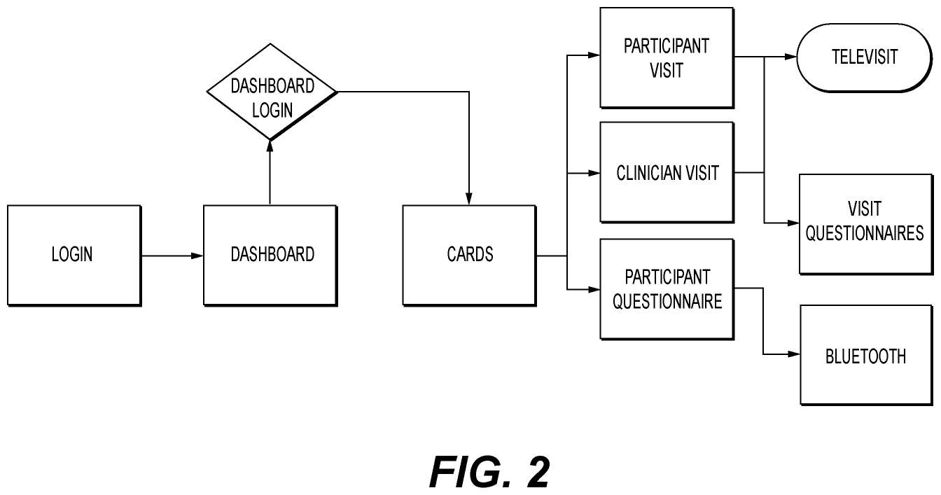

FIG. 2 depicts an exemplary flowchart of a patient accessing a study, according to one or more embodiments. FIG. 2 may, for example, depict devices or aspects of a dashboard that may be utilized by a mobile device. For example, in some embodiments, the “participant questionnaire” and “visit questionnaire” are interfaces that a clinical study patient may interact with through a mobile interface.

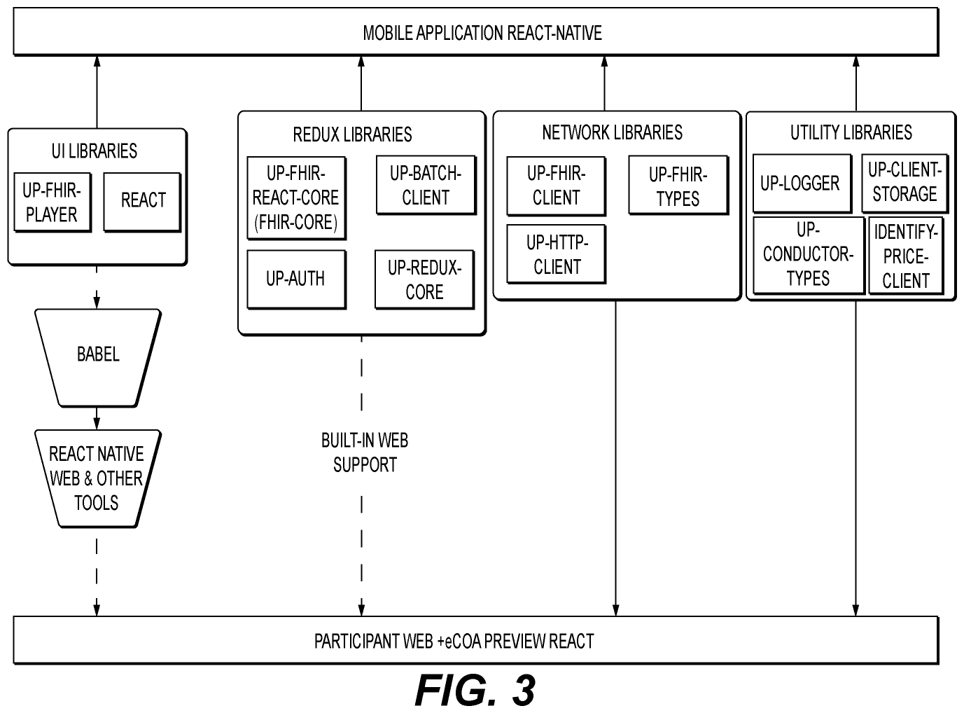

FIG. 3 depicts a dependency stack of the previewer 123, according to one or more embodiments. FIG. 3 depicts the object map of proxy object utilized by the previewer 123 to, for example, provide a preview to developers of a clinical questionnaire (e.g., eCOAs) during development of the eCOA. This may allow for the rendering and review of screenshots of a particular instrument (e.g., a eCOA) during preparation of the particular instrument.

FIG. 3 illustrates the different library repositories of code utilized for a web interface and for a mobile interface. Conventionally, several libraries do not natively support operation on a web interface. For example, the UI Library may rely on secondary tools to adapt the libraries for web use. Such layering may result in differing results for the display of the accessed library in an interface, depending on how the application is accessed. However, by directly integrating the application, such issues may be avoided. In an example, a direct integration may enable both a web and mobile interface to both work with previews and utilization of the eCOAs prepared by the system herein. They may further allow the user to preview the eCOA through a web interface configured to display a mobile interface. In one example, Babel and React Native for Web software may assist with translating the code from a web interface to a mobile interface. In another example, the Babel and React Native for Web software may not be required to translate the code from a web interface to a mobile interface.

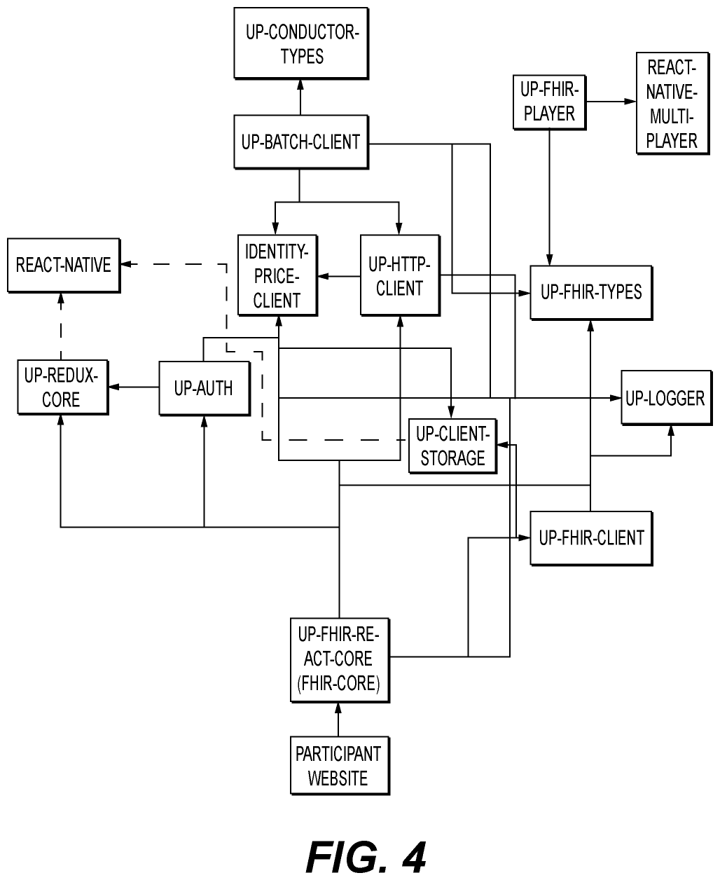

FIG. 4 depicts a dependency diagram of the previewer 123, according to one or more embodiments. FIG. 4 may, for example, depict the dependencies of particular libraries accessed by the system described herein. For example, this may illustrate which internal libraries utilized by the system described herein have particular dependencies.

As discussed above, the server systems 108 may include direct integration of the preview function, allowing for scalable access to the eCOA library (e.g., by an administer creating and previewing a validated instrument, and by a user accessing a validated instrument). By utilizing “direct integration”, the system may directly integrate the website's code into a device's (e.g., a mobile device's) operating system. This may allow for a more efficient and seamless experience for users accessing the device. Further, the use of direct integration may allow for the same engine to provide the preview as the engine that outputs the product.

For example, an administrator may run a preview of an eCOA that is in the middle of production. The system described herein may interact with one or more UI libraries, libraries for managing and centralizing the state of an application (e.g., Redux libraries), network libraries, and/or utility libraries to prepare an eCOA preview through the web, of a mobile application. The system may utilize the same directly integrated players to provide access for a user to the finalized versions of the eCOA.

In the following methods, operations are described that may be performed by one or more of the devices in the environment 100 of FIG. 1. It should be understood that the follow methods are illustrative, and that, in various embodiments, one or more steps may be added, removed, and/or rearranged.

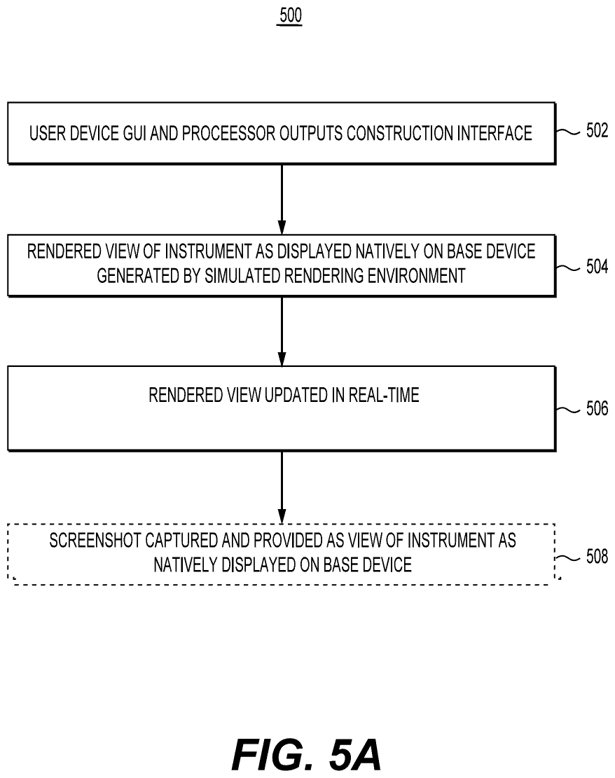

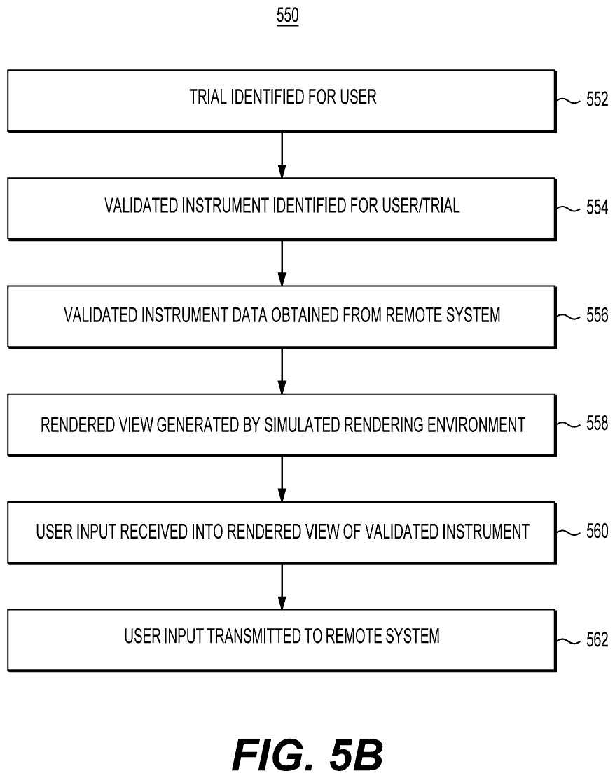

FIG. 5A depicts an exemplary method 500 for providing a device-agnostic environment with real-time rendering during instrument construction, according to one or more embodiments.

At step 502, a processor of a user device may cause a graphical user interface of the user device to output a construction interface. The interface may be operable to edit contents of an instrument, including, for example, an electronic clinical outcome assessment (eCOA), a Participant Questionnaire, or Visit Questionnaire, which may, in certain embodiments, be comprised of a plurality of parts. In some embodiments, the graphical user interface of the user device is a web interface.

At step 504, the processor may employ a simulated rendering environment of a to generate a rendered view of the instrument. The simulated environment may, for example, be operating on the user device, and may be configured to interpret instrument data as if it were being displayed natively on a base device, ensuring a device-agnostic representation. In some embodiments, the processor employs a simulated rendering environment of a mobile device to generate a rendered view of the instrument, which may be output on an interface, e.g., a GUI, which may also be a web interface. The rendered view of the instrument may be interactive, e.g., may enable navigation between separate parts (e.g., pages or sections), may include interactive elements such as fields or interface elements for entering replies. The rendered view may simulate or access back-end resources that operate with an instrument in a production environment. For instance, an instrument may access external data and/or transmit response data to a data store, and the rendered view may simulate and/or enable such operations, e.g., by actually providing access to such resources and/or a simulation thereof.

At 506, the simulated rendering environment may update the rendered view in real-time in response to adjustment to the construction interface. For example, the user 102 may use the construction interface to navigate to a portion of the instrument, or make an adjustment to the instrument. Further, the user 102 may interact directly with the instrument, e.g., to enter a response, or the like, e.g., in order to test functionality. In some embodiments, the updates to the rendered view are in response to adjustments to the construction interface as a result of interaction with interactive elements of the construction interface. In some embodiments, interaction with the construction interface edits contents of an instrument.

In some embodiments, at a step 508, a screenshot of the rendered view may be captured. The screenshot may be provided for representing a view of the instrument as if the instrument were natively displayed on the base device.

FIG. 5B depicts an exemplary method 550 for providing a device-agnostic environment for displaying validated instruments, according to one or more embodiments.

At step 552, an identification of a trial associated with a user of the user device may be received. At step 554, a validated instrument may be identified as corresponding to a user and/or to the trial, e.g., by accessing a remote system (e.g., the processing devices 110 of FIG. 1).

At step 556, the data of the validated instrument may be obtained from the remote system. In some embodiments, the data is for an electronic clinical outcome assessment (eCOA), a Participant Questionnaire, or Visit Questionnaire, and may comprises a plurality of parts.

At step 558, a rendered view of the validated instrument based on the obtained data may be output to a graphical user interface of the user device. The rendered view may be generated by a simulated rendering environment, e.g., operating on a user device, of a base device such that the simulated rendering environment interprets data of the validated instrument and generates the rendered view as if the validated instrument were natively displayed on the base device and while being agnostic to display characteristics of the user device. In an embodiment, the base device is a mobile device, with the rendered view being rendered on the user device by a simulated rendering environment of a mobile device operating on the user device.

At step 560, user input may be received into the rendered view of the validated instrument, e.g., via the graphical user interface. At step 562, the user input may be transmitted to the remote system.

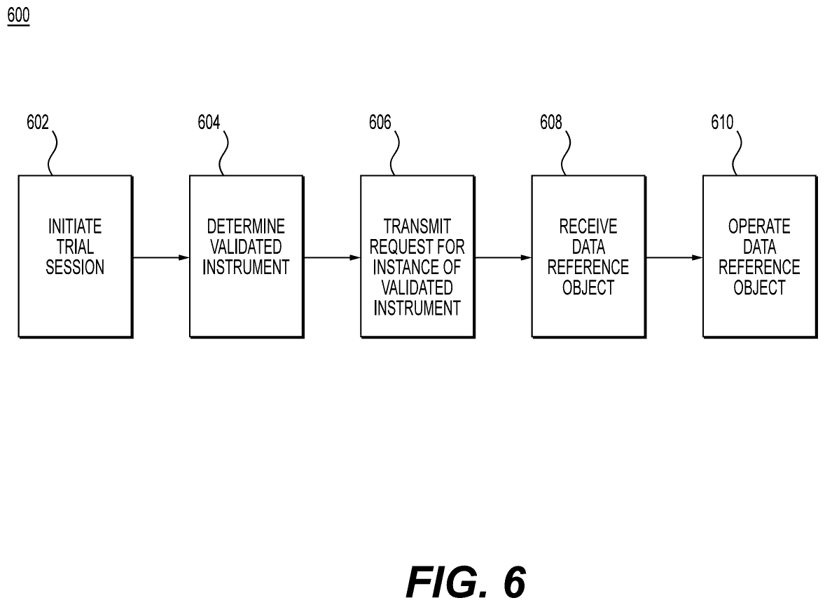

FIG. 6 depicts an exemplary flow diagram illustrating a workflow for obtaining an instances of a validated instrument, according to one or more embodiments. The flow diagram described in FIG. 6 may be performed by the environment 100 of FIG. 1. In other examples, aspects of the flowchart described in FIG. 6 may be performed in external systems and received by the environment 100. Alternatively, the flow diagram described in flowchart 600 may be performed by any computer process system capable of receiving image inputs such as computer 800.

At step 602, a trial session for a user may be initiated. A trial session may refer to the process and systems for a particular user to complete an instrument associated with a particular clinical study. For example, a trial session for a user may be initiated once the user has completed a portion of a clinical study. The trial session may be initiated by a provider of the clinical study. The trial session may be initiated to help standardize a clinical endpoint to meet specific measures set forth to support changes in reported data. The trial session may for example be initiated by an eCOA manager 104 accessing an electronic network 106 in order to allow a user 102 to access an eCOA from the eCOA library 120. The trial server may be initiated within the processing devices 110 of FIG. 1. Initiating the trial session may include associating the user with a study, wherein the validated instrument is associated with the study. The user may further be associated with a particular language or locale. The user may access the trial server via an authorization system (e.g., authorization system 103), with the authorization system including (i) an identity provider web service and/or a system configured to access a fast healthcare interoperability resource server (e.g., where the eCOAs may be located within the eCOA library 120), and (ii) a firewall.

At step 604, the system (e.g., environment 100 of FIG. 1) may determine, via the trial server (e.g., via the processing devices 110), a validated instrument to be accessed for the trial session. For example, the validated instrument may be an eCOA. The validated instrument may for example be determined by extracting what clinical study a user is involved in. For example, the validated instrument may be extracted from the studies module 116 or a stored schedule for a clinical study.

At step 606, a request for an instance of the validated instrument may be transmitted to an instrument library (e.g., the eCOA library 120) and via the trial session (e.g., the processing devices 110). The instance of the validated example, may be a reference or pointer to a previously validated instrument. The instance of the validated example may be a validated screenshot of the validated instrument, allowing for the user to insert answers into the validated screenshot.

At step 608, a data reference object that is operable as a data reference link to the validated instrument stored in the instrument library (e.g., the eCOA library 120) is received from the instrument library, such that the data reference object is operable to execute the validated instrument without generating a copy of the validated instrument.

At step 610 a Graphical User Interface (GUI) of the trial session may operate the data reference object, such that a user device (e.g., device 700 as described in more detail below) executes the validated instrument by reference. This may allow one or more users (e.g., user 102) to access the validated instrument through a GUI (e.g., by accessing the server systems 108 through electronic network 106) and the techniques of, e.g., FIG. 5B, may be applied to allow one or more users to interact and with and respond to the instrument. The results may then be saved to one or more storage devices (e.g., storage devices 112).

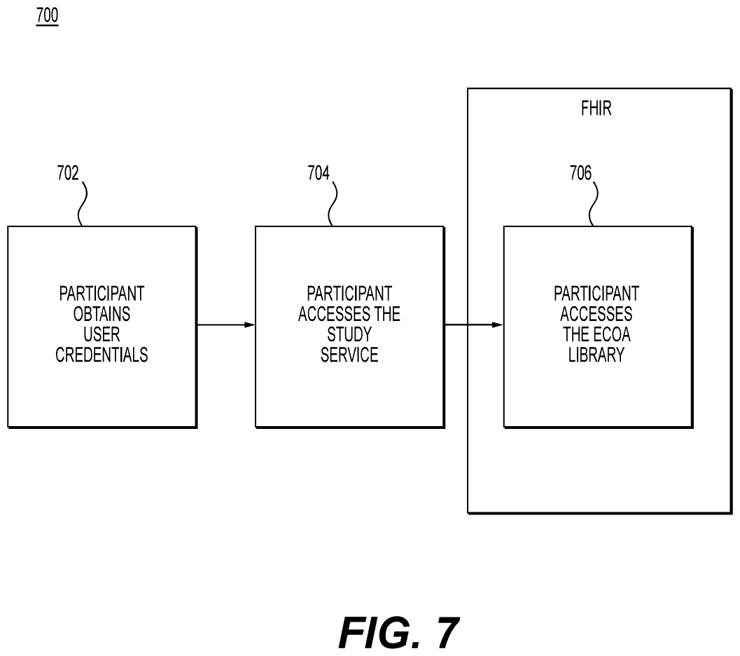

FIG. 7 depicts an exemplary flow diagram illustrating a workflow for obtaining access to a validated instrument, according to one or more embodiments. The flow diagram described in FIG. 7 may be performed by the environment 100 of FIG. 1. In other examples, aspects of the flowchart described in FIG. 7 may be performed in external systems and received by the environment 100. Alternatively, the flow diagram described in flowchart 300 may be performed by any computer process system capable of receiving image inputs such as computer 700.

At step 702, a participant may obtain user credentials in order to provide initial access to a study. For example, a patient may be introduced to a clinical study. For example, a patient may be selected for a study that a particular organization operates. Upon being selected, a patient may be administered a particular care plan. The care plan may be patient specific or generated evenly for a study. The patient may, for example, go to organization site to be a participant in the clinical study. The organization site may, for example, have a medical equipment to administer the collection of samples, reading, etc. In another example, the clinical study may be administered in a remote fashion across a large geographical area with a large sample size of patients. A first step may be to provide the participant with credentials to access the study. For example, a user may be assigned (e.g., associated) with multiple credentials (e.g., a password, code, etc.) where the credential may be associated with a particular user. The credential may be assigned at an organization cite or online (e.g., through network 106). The credentials may allow a user to access one or more portions or aspects of the server systems 108 of FIG. 1. For example, the credentials may allow for a user to access data submitted from a particular organization, data such as questionnaires from the eCOA library 120.

At step 704, the participant may access the study service (e.g., a particular organization's study). The organization study may for example be administered by an organization sponsor, where the organization sponsor administer the clinical study through a secondary organization. While accessing the organization study, the patient may follow part of a previously developed plan for the clinical trial. The plan may for example have been created and may be administered by the server systems 108.

At step 706, the participant may access the eCOA library (e.g., the eCOA library 120). For example, this access may occur upon completion of a portion of the clinical trial, whereby the research may include a clinical endpoint to meet specific measures set forth to support changes in reported data. For example, a patient (or alternatively, an administer of the clinical study on behalf of the patient) may request authorization of an instrument. For example, a user (e.g., the user 102) may utilize an authorization system (e.g., the authorization system 103) through a network to request access to the eCOA library. Upon receiving approval, a user may first complete a contract or fill out identifying information. Next, the user may obtain access to an eCOA (e.g., through the eCOA library 120). The eCOA library may for example be stored in a FHIR server. The eCOA may, for example, be a data reference object that is operable as a data reference link to the validated eCOA in the eCOA library. Next, using the techniques of, e.g., FIG. 5B, the user may utilize the previewer 123 and interact with a GUI to fill out the questionnaire. The answers may then be saved to storage (e.g., storage devices 112) for further analysis (e.g., by the report and analytics module 122). In certain alternate embodiments, the user may not be a participant, and may, using the techniques of e.g., FIG. 5A, utilize the previewer 123 to access the eCOA. In various embodiments, the user may be a developer and/or administrator, and may access the eCOA. In certain embodiments, a user utilizing the previewer 123 may edit the contents of an eCOA or the eCOA library.

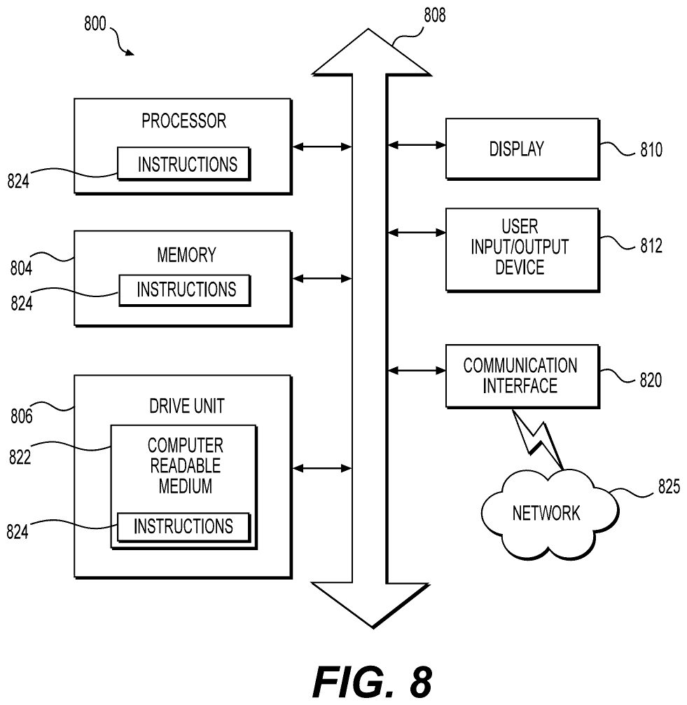

FIG. 8 depicts a simplified functional block diagram of a computer, according to one or more embodiments. The computer 800 of FIG. 8 may be utilized by a user 102 or an eCOA manager 104 to access the server systems 108 of FIG. 1. Further the computer 800 may be utilize to execute the methods of FIG. 4, 5, 6, or 7 according to exemplary embodiments of the present disclosure. One or more of a processor 802, a memory 804, a drive unit 806, an internal communication bus 808, a display 810, a under input/output ports 812, a communication interface 820, a computer readable medium 822, instructions 824, and a network 220 may communicate by any suitable means. For example, computer 800 may be configured as the one or more sensors 105, patient communication device 205, and/or another system according to exemplary embodiments of this disclosure. In various embodiments, any of the systems herein may be a computer 800 including, for example, data communication interface 820 for packet data communication. Computer 800 also may include a central processing unit (CPU) 802, in the form of one or more processors, for executing program instructions. Computer 800 may include internal communication bus 808, and storage unit 806 (such as Read-Only Memory (ROM), Hard Disk Drive (HDD), Solid-State Drive (SSD), etc.) that may store data on computer readable medium 822, although computer 800 may receive programming and data via network communications. Computer 800 may also have memory 804 (such as Random-Access Memory (RAM)) storing instructions 824 for executing techniques presented herein, although instructions 824 may be stored temporarily or permanently within other modules of computer 800 (e.g., processor 802 and/or computer readable medium 822). Computer 800 also may include input and output ports 812 and/or display 810 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, medical equipment, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments may be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for providing a device-agnostic environment with real-time rendering during instrument construction, the computer-implemented method comprising:
via a processor of a user device, causing a graphical user interface of the user device to output a construction interface operable to edit contents of an instrument; and
via the processor, causing the graphical user interface to output a rendered view of the instrument, wherein:
the rendered view is generated via a simulated rendering environment, operating on the user device, of a base device such that the simulated rendering environment interprets data of the instrument and generates the rendered view as if the instrument were natively displayed on the base device and while being agnostic to display characteristics of the user device; and
the simulated rendering environment is configured to update the rendered view in real-time in response to adjustment to the construction interface.

2. The computer-implemented method of claim 1, wherein the construction interface includes interactive elements, and wherein interacting with the interactive elements of the construction interface causes real-time changes to the rendered view of the instrument.

3. The computer-implemented method of claim 1, wherein the construction interface includes interactive elements, and wherein interacting with the interactive elements of the construction interface edits contents of the instrument.

4. The computer-implemented method of claim 1, wherein the instrument comprises a plurality of parts, and the rendered view comprises at least one of the plurality of parts.

5. The computer-implemented method of claim 1, wherein the base device is a mobile device.

6. The computer-implemented method of claim 1, wherein the graphical user interface of the user device is a web interface.

7. The computer-implemented method of claim 1, wherein the instrument includes one or more of an electronic clinical outcome assessment (eCOA), a Participant Questionnaire, or Visit Questionnaire.

8. The computer-implemented method of claim 1, further comprising:
capturing at least one screenshot of the rendered view of the instrument during instrument construction; and
providing the at least one screenshot for representing a view of the instrument as if the instrument were natively displayed on the base device.

9. A computer-implemented method for providing a device-agnostic environment for displaying validated instruments, the computer-implemented method comprising:
via a processor of a user device, receiving an identification of a trial associated with a user of the user device;
via the processor, identifying a validated instrument corresponding to the trial and to the user by accessing a remote system;
via the processor, obtaining data of the validated instrument from the remote system;
via the processor, causing a graphical user interface of the user device to output a rendered view of the validated instrument based on the obtained data, wherein the rendered view is generated via a simulated rendering environment, operating on the user device, of a base device such that the simulated rendering environment interprets data of the validated instrument and generates the rendered view as if the validated instrument were natively displayed on the base device and while being agnostic to display characteristics of the user device;
receiving, via the processor and by the graphical user interface, user input into the rendered view of the validated instrument; and
via the processor, transmitting the user input to the remote system.

10. The computer-implemented method of claim 9, wherein the validated instrument comprises a plurality of parts, and the rendered view comprises at least one of the plurality of parts.

11. The computer-implemented method of claim 10, wherein the graphical user interface includes an interactive element operable to cause the rendered view to switch between the plurality of parts.

12. The computer-implemented method of claim 9, wherein the base device is a mobile device.

13. The computer-implemented method of claim 9, wherein the graphical user interface of the user device is a web interface.

14. The computer-implemented method of claim 9, wherein the validated instrument includes one or more of an electronic clinical outcome assessment (eCOA), a Participant Questionnaire, or Visit Questionnaire.

15. A computer-implemented method for providing a device-agnostic environment for displaying instruments, the computer-implemented method comprising:
via a processor of a user device, obtaining data of an instrument;
via the processor, executing a simulated rendering environment that simulates a native display of a base device while being agnostic to display characteristics of the user device;
via the processor, causing a graphical user interface of the user device to output, using the simulated rendering environment, a rendered view of the instrument based on the obtained data, such that the instrument is displayed as if the instrument were natively displayed on the base device.

16. The computer-implemented method of claim 15, wherein:
the graphical user interface includes interactive elements; and
the interactive elements of the graphical user interface are operable to cause changes to the rendered view of the instrument.

17. The computer-implemented method of claim 15, further comprising:
receiving, via the processor and by the graphical user interface, user input into the rendered view of the instrument; and
via the processor, transmitting the user input to a remote system.

18. The computer-implemented method of claim 1, wherein the graphical user interface of the user device is a web interface.

19. The computer-implemented method of claim 1, wherein the instrument comprises a plurality of parts, and the rendered view comprises at least one of the plurality of parts.

20. The computer-implemented method of claim 1, further comprising:
capturing at least one screenshot of the rendered view of the instrument during instrument construction; and
providing the at least one screenshot for representing a view of the instrument as if the instrument were natively displayed on the base device.
